(19) United States
(12) Patent Application Publication
(10) Pub. No.: US 2008/0241836 A1
(43) Pub. Date: Oct. 2, 2008

(54) PROCESS FOR SELF-ASSEMBLY OF STRUCTURES IN A LIQUID

(76) Inventors: Gafur Zainiev, West Bloomfield, MI (US); Inlik Zainiev, West Bloomfield, MI (US); Timur Zainiev, West Bloomfield, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/927,891

(22) Filed: Mar. 31, 2008

Related U.S. Application Data
(63) Continuation-in-part of application No. 11/835,054, filed on Aug. 7, 2007.
(60) Provisional application No. 60/836,103, filed on Aug. 7, 2006.
(60) Provisional application No. 60/905,357, filed on Mar. 7, 2007.

(51) Int. Cl. C12Q 1/68; C12P 19/34; C12M 1/00

(52) U.S. Cl. 435/6; 435/91.1; 435/287.2

(57) ABSTRACT

A process and apparatus for self-assembling a number of elements and determining their sequence is provided. In the field of DNA analysis, an iterative process is disclosed wherein an apparatus with a set of reaction chambers in which a species of recognition element nucleotides are differentially added and subjected to a polymerization reaction allows recognition of which species is next in sequence on a template strand by the effect that synthesis has on a detecting template as measured by a detector in a detection area. Stepwise addition of the identified species then determines if an element repeat exists. The process is repeated until the entire structure is complete and the sequence identified.

### CROSS-REFERENCE TO RELATED APPLICATIONS

This application is a continuation-in-part of the U.S. patent application Ser. No. 11/835,054 filed Aug. 8, 2007, which claims the benefit of U.S. Provisional Patent Applications 60/836,103 filed Aug. 7, 2006; and 60/905,357 filed Mar. 7, 2007.

### FIELD OF THE INVENTION

The present invention relates to the field of self-assembly of a number of elements into a structure. More particularly, the present invention relates to the assembly of nucleotides to form an oligonucleotide structure and sequence determination thereof. Most particularly, this present invention relates to the field of DNA sequencing.

### BACKGROUND OF THE INVENTION

The art of DNA sequencing, long accomplished by a multi-step brute force approach, was radically transformed by the development of new technologies during the human genome project advancing the pace of sequencing a genome from years to months. Completion of the human genome project saw successful innovations in the fields of recombinant protein engineering, fluorescent dyes, capillary electrophoresis, automation, informatics and process management. (Metzger, M. L., Genome Res, 2005; 15:1767-76).

Modern sequence analysis is most commonly directed toward discovery and analysis of sequence variation as it relates to human health and disease. These continue to be large-scale projects that are plagued by technology that is slow in its application and inaccurate in its nature. Further, current technologies available for sequence analysis tend to require large amounts of nucleic acid template and large biological samples. Important parameters which can be addressed by improved technology include increased sequencing speed, increases in sequence read length achievable during a single sequencing run, decreasing in the amount of template required to obtain positive sequence results, decreasing the amount of reagent required for processing a sequence reaction, improving the accuracy and reliability of the sequences generated, and improved identification of nucleic acid repeats in the strand of DNA.

Several unique approaches are traditionally employed for sequencing DNA. The most common is the dideoxy-termination method of Sanger (Sanger et al., PNAS USA, 1977; 74:563-567). Single nucleotide analysis such as pyro-sequencing first described by Hyman 1988 (Analytical Biochemistry, 174, pages 423-436) has proved to be the most successful non-Sanger method. Cyclic reversible termination or CRT has also been employed with some success. Finally, sequence analysis has been accomplished by an exonuclease reaction wherein particular nucleotide residues are identified in a stepwise fashion as they are removed from the end of an oligonucleotide strand.

The Sanger method represents a mixed mode process coupling synthesis of a complementary DNA template using deoxynucleotides (dNTPs) with synthesis termination by the use of fluorescently labeled dideoxynucleotides (ddNTPs). Balancing reagents between natural dNTPs and ddNTPs leads to the generation of a set of fragments terminating at each nucleotide residue within the sequence. The individual fragments are then detected following capillary electrophoresis so as to resolve the different oligonucleotide strands. The sequence is determined by identification of the fluorescent profile of each length of fragment. This method has proven to be both labor and time intensive and requires extensive pretreatment of the DNA source. Microfluidic devices for the separation of resulting fragments from Sanger sequencing has improved sample injection and even decreased separation times, hence, reducing the overall time and cost of a DNA sequencing reaction. However, the time and labor required to successfully prosecute a Sanger method is still sufficiently great to make several studies beyond the reach of many research labs.

The single nucleotide addition methodology of pyro-sequencing has been the most successful non-Sanger method developed to date. Pyro-sequencing capitalizes on a non-fluorescence technique, which measures the release of inorganic phosphate converted to visible light through a series of enzymatic reactions. This method does not depend on multiple termination events, such as in Sanger sequencing, but instead, relies on low concentration of substrate dNTPs, so as to regulate the rate of dNTP synthesis by DNA polymerase. As such, the DNA polymerase extends from the primer, but pauses when a non-complementary base is encountered until such time as a complementary dNTP is added to the sequencing reaction. This method, over time, creates a pyrogram from light generated by the enzymatic cascade, which is recorded as a series of peaks and corresponds to the order of complementary dNTPs incorporated revealing the sequence of the DNA target. (See Ronaghi, Science, 1998; 281:363-65; Ronaghi, Analytical Biochemistry, 2002; 286:282-288; Langaeet and Ronaghi, Mutational Research, 2005; 573:96-102). While pyro-sequencing, has the potential of reducing sequencing time, as well as amount of template required, it is typically limited to identifying 100 bases or less. Further, repeats of greater than five nucleotides are difficult to quantitate using pyro-sequencing methods. Also, pyro-sequencing methods must be carefully designed, as it is the order of dNTP addition that determines the pyrogram profile and investigators must design experiments so as to avoid asynchronistic extensions of heterozygous sequences as almost half of all heterozygous sequences result in asynchronistic extensions at the variable site. (Metzger, 2005).

Cyclic Reversible Termination (CRT) uses reversible terminating deoxynucleotides, which contain a protecting group that serves to terminate DNA synthesis. A termination nucleotide is incorporated, imaged, and then deprotected so that the polymerase reaction may incorporate the next nucleotide in the sequence. CRT has advantages over pyro-sequencing in that all four bases are present during the incorporation phase, not just a single base during a single period of time. Single base addition is achievable through homopolymer repeats and synchronistic extensions are easily maintained past heterozygous bases. Perhaps the greatest advantage of CRT is that it may be performed on many highly parallel platforms, such as high-density oglionucleotide arrays (Pease et al., 1994, and Albert et al., 2003), PTP arrays (Laymon et al., 2003), or random dispersion of single molecules (Nutra and Church, 1999). High-density arrays and incorporation of di-labeled dideoxynucleotide dNTPs by DNA polymerase gives CRT significant improvement in throughput and accuracy. However, CRT suffers several drawbacks including short read lengths that must be overcome before it can be widely employed.

Finally, exonuclease methods sequentially release fluorescently labeled bases as a second step following DNA polymerization to a fully labeled DNA molecule. Using a hydrodynamic flow detector, each dNTP analog is detected by its fluorescent wavelength as it is cleaved by the exonuclease. This method has several drawbacks. For example, the DNA polymerase and, more importantly, the exonuclease must have high activity on the modified DNA strand and generation of a DNA strand fully incorporating four different fluorescent dNTP analogs has yet to be achieved.

Technological advances in fluorescence detection are essential to decrease the amount of target oglionucleotide necessary for sequencing analysis. Four color fluorescent systems such as those employed in Sanger methods have several disadvantages including inefficient excitation of fluorescent dyes, significant spectra overlap between each of the dyes, and inefficient collection of the emission signal. Several dyes have been recently developed that help address these issues, such as fluorescence resonance energy transfer (FRET) dyes (Ju et al., PNAS, 1995; 92:4347-51; Metzger, Science, 1996: 271:1420-1422.) Additional strategies have been proposed, such as fluorescence lifetime and a radio frequency modulation. Finally, Lewis et al. recently described termed pulse multiline excitation (PME) which is an ineffective method for multifluorescence discrimination. (Lewis, PNAS, 2005: 102:5346-41).

The demand for rapid small and large scale DNA sequencing has radically increased over the last several years. Current sequencing methods tend to be expensive and time consuming. Further, the prior art methods each suffer the drawback of inaccuracy in identification of repeat nucleotides in the sequence. Thus, there remains a need for a rapid and accurate sequencing method that can be run on an automated platform.

### SUMMARY OF THE INVENTION

The present process relates to the assembly of structures comprised of a number of elements in a solution wherein the structure is complementary to a target strand. The inventive process is accomplished by providing a set of N recognition chambers, each chamber divided into a reactor area and a detection area. A plurality of sequencing and detecting templates is added to each chamber. A detecting template is either a homopolymeric or heteropolymeric sequence. Subsequently, a plurality of recognition elements is added into each chamber with each chamber receiving a homogeneous species of recognition elements that are distinguishable from the recognition elements added to each of the other chambers. However, a chamber array is optionally created wherein all recognition element species are delivered simultaneously to each chamber. The sequencing template and recognition elements are then subjected to a polymerization reaction with a plurality of polymerization enzyme so that a complementary recognition element binds and is assembled into the final structure. The process continues by identifying which of the recognition elements is complementary by method of subtraction or by detecting the effect on synthesis of double stranded DNA in a detection area on detecting template. Finally, a plurality of building elements, each building element corresponding to the incorporated recognition element, is added to each of N chambers to complete the addition of elements at that step. This sequence is repeated until the structure is complete. By identification of each of the individual elements as it is added to the growing oligomeric structure, the sequence of the template is determined. In a nonlimiting example, if the number of elements in the sequence is 4, N is 4.

A detecting template is optionally immobilized or is solution in either or both of the reactor area or detection area. Optionally, a capture agent is immobilized that specifically recognized double stranded DNA. The capture agent is a DNA transcription factor, mismatch repair protein, double stranded DNA recognizing antibody, peptide nucleic acid, a DNA intercalator, precursors of any of the previous, cleavage products of any of the previous, or a nullity.

The sequencing and detecting templates are optionally immobilized on a support or free in solution.

Optionally, all of the recognition elements are washed away from each of the recognition chambers prior to addition of building elements. Recognition elements and building elements are optionally selected from the group including nucleotides, ribonucleotides, deoxynucleotides, dideoxynucleotides, peptide nucleotides, modified nucleotides, modified peptide nucleotides, modified phosphate sugar backbone nucleotides, amino acids, or modified amino acids.

In addition to the recognition chambers, the process optionally employs a repeat detection chamber. Repeat detection achieved by small stepwise addition of less than saturated amounts of building or recognition element and detection of free element following each stepwise addition. When the element added is no longer placed in sequence, the particular site on the template is considered saturated and the number of repeat elements in the sequence is calculated.

It is further envisioned that the liquid solution from each of the recognition chambers is optionally transferred to the repeat detection chamber prior to addition of recognition or building elements. After repeat detection, the liquid reaction material is then optionally transferred from the repeat detecting chamber and divided among all N recognition chambers prior to addition of recognition elements. Optionally, all the recognition or building elements are washed out of the repeat detecting chamber or the recognition chambers prior to addition of further elements.

In an alternative embodiment, a sequence construction chamber is additionally employed where solution from the recognition chambers and the repeat detection chamber is transferred to the sequence construction chamber and the structure is increased by addition of complementary building elements. Optionally, a large sequence construction chamber is employed so that after each step of building element addition, a volume of liquid is transferred from the sequence construction chamber back to the repeat detecting chambers, as well as each of the recognition chambers. This template is then added to by new recognition elements to determine what the next element and sequence is.

It is appreciated that the oligionucleotide, or ogligomeric template, is immobilized on a support, or free in solution. It is further appreciated that recognition or building elements are optionally washed away and removed from each of the N recognition chambers, the repeat detecting chamber, or the sequence construction chamber so that a clean template can be reutilized upon each subsequent addition, hence regenerating the system. The polymerization enzyme responsible for the polymerization reaction is illustratively a DNA polymerase, an RNA polymerase, a reverse transcriptase, or mixtures thereof.

As opposed to the template being attached to a support, the polymerization enzyme is optionally attached to the support, or is itself free in solution. The nucleic acid polymerizing enzyme is optionally a thermostable polymerase or a thermodegradable polymerase. Template types operable in the instant invention include double-stranded DNA, single-stranded DNA, single-stranded DNA hairpins, RNA, and RNA hairpins. The template is optionally attached to a support by hybridizing to a primer sequence that is itself optionally affixed to a support. The primer sequence is free in solution and is complementary to a small segment of the target sequence so that a polymerization reaction may be extended from the primer. The primer is optionally covalently hybridized to the sequencing or detecting template.

Recognition elements optionally comprise a label or a plurality of labels and a protecting group. Numerous label types are operable in the instant invention illustratively including chromophores, fluorescent moieties, haptens, enzymes, antigens, dyes, phosphorescent groups, chemiluminescent moieties, scattering or fluorescent nanoparticles, FRET donor or receptor molecules, Raman signal generating moieties, precursors thereof, clevage products thereof, and combinations thereof. In addition, photobleachable, photoquenchable, or otherwise inactivatable labels are similarly operable. The label or protecting group is optionally attached to a recognition element at any suitable site illustratively including a base, a sugar moiety, an alpha phosphate, beta phosphate, gamma phosphate, or combinations thereof. It is appreciated that each homogeneous species of recognition element optionally carries a label that is distinguishable from other labels on different recognition elements.

Detection of free recognition element is accomplished by one or many of numerous identifying techniques; illustratively, far field microscopy, near field microscopy, evanescent wave or wave guided illumination, nanostructure enhancement, photon excitation, multiphoton excitation, FRET, photo conversion, spectral wavelength discrimination, fluorophore identification, background suppression, mass spectroscopy, chromatography, electrophoresis, surface plasmon resonance, enzyme reaction, fluorescence lifetime measurements, radio frequency modulation, pulsed multiline excitation, or combinations thereof.

Background fluorescence or fluorescence of previously added recognition elements to a growing structure is optionally eliminated by photobleaching the label, cleaving the label, or otherwise inactivating the label. The label is optionally cleaved from the backbone prior or subsequent to addition of recognition or building elements.

The present invention also envisions an apparatus for self-assembly of a number of elements into a structure that comprises a reaction area, a preparation area, which is in fluidic connection with said reaction area, and a detection area, which is in fluidic, physical, or optical connection with the reaction area or preparation area. It is appreciated that the reaction area has no moving parts. Within the reaction area there are N recognition chambers where each chamber has a plurality of microdispensers. Each microdispenser is capable of dispensing a unique species of recognition element or a building element. Optionally, each dispenser is employed to dispense numerous elements or polymerization components simultaneously, or be washed out between the dispensing of a particular element. The chambers within the reaction area are optionally a batch flow reactor, a plug flow reactor, or a drop reactor.

### BRIEF DESCRIPTION OF THE DRAWINGS

The present invention is further detailed with respect to the following nonlimiting figures. These figures depict only particular processes and apparatuses according to the present invention with variants existing beyond those depicted.

FIG. 1A is a schematic of a set of reaction chambers that contain reaction chamber solution 4, template molecule 3, polymerizing enzyme 5, and all other necessary reagents wherein the open site on the template molecule 3 is depicted by a T such that only the chamber with the A recognition element 1 is subjected to a successful polymerization reaction removing the A recognition element from the solution of that chamber alone and allowing detection of free recognition element species 1 in all other chambers leading to identification of which nucleotide species is in the hybridization position;

FIG. 1B1 depicts a primer 6 affixed to a support 7, the primer 6 hybridized to a DNA template molecule wherein a polymerizing enzyme 5 recognizes the 3′ end of the template 3, and the amount of recognition element 1 added to the reaction chamber is less than that required to saturate all hybridization sites on the template strands 3;

FIG. 1B2 depicts alternative schematic immobilization of FIG. 1B1 relative to a support wherein DNA template molecule affixed to a support 7, the DNA template molecule hybridized to a primer 6 wherein a polymerizing enzyme 5 recognizes the 3′ end of the template 3, and the amount of recognition element 1 added to the reaction chamber is less than that required to saturate all hybridization sites on the template strands 3;

FIG. 1C depicts a two reaction chamber protocol wherein a biotin/streptavidin interaction immobilizes a primer 6 to a support 7 and hybridization of the template 3 to the primer immobilizes the template to the support, hence, creating a binding site for a polymerizing enzyme 5 such that a complementary recognition element depicted by a rectangle is able to hybridize with the open site on the template molecule and the polymerizing enzyme binds the complementary recognition element to the growing primer strand to form the structure, whereas the non-homologous recognition element depicted by a triangle in chamber 2 will not be added to the growing structure, furthermore, stepwise addition of the complimentary recognition element species of building element completes the repeat determination step and saturation of all template strands, washing out of all unbound elements allows a repeat of the procedure in the same reaction chambers to complete the assembly and sequence identification of the structure;

FIG. 1D depicts a schematic of chambers 1 and 2 of FIG. 1C wherein an electric potential applied throughout a detection chamber 19 is used to selectively move non-complementary elements from the reaction chamber past a detector to a collection area 18; the collected free recognition elements are optionally returned to the reaction chamber by reversing the polarity of the electric field;

FIG. 2 depicts an alternative embodiment of a reaction chamber 2 wherein recognition elements 1 are flowed over template/primer/polymerase immobilized on a support 7 mediated by a pump 16 and the presence of free recognition element 1 is determined by a detector 9;

FIG. 3 depicts an overall schematic for an apparatus of the instant invention that includes a reaction area 17 that contains reaction chambers 2 each with a plurality of microdispensers 8 that dispense elements into the reaction chamber solution 4, and the reaction chamber is in fluid connection by a fluid communication medium 12 to a repeat detecting chamber 10 and a sequence building chamber 11, the reaction area 17 is in connection with a detection area 14 and a reagent preparation area 13. In a non-limiting example where N is 4, each microdispenser 8 supplies only one homogenous type of mono nucleotide such as A, T, G or C;

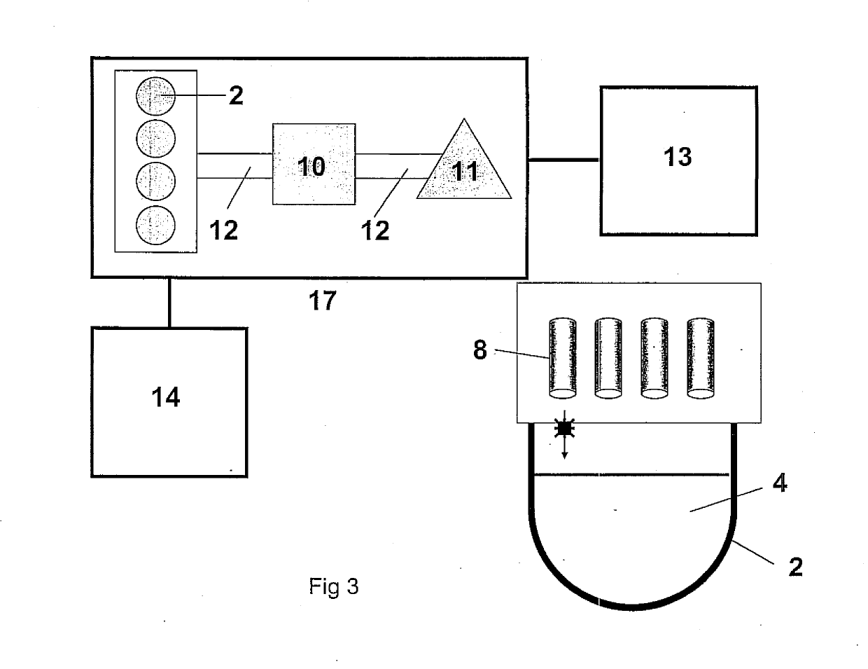

FIG. 4 depicts a schematic reaction chamber with a reactor area 21 and a detection area 14 in fluidic connection by a medium 12 such that sequencing template is immobilized in the reactor area 21 into which a single or plurality of microdispensors 8 adds elements into the reactor area 21 allowing a polymerization reaction to begin followed by application of an electric potential (depicted by the + and − signs) to move any unbound recognition elements from the reactor area 21 to a detecting area 14 where a second set of polymerization reactions occurs so that strand synthesis only occurs on the respective detecting template if the next in sequence element is not the element that was added to the reactor area such that a detector 9 will identify the next in sequence element; the detector area is regenerated by heating by a heat block 20 so as to melt the double stranded DNA and a washing procedure removes the non immobilized strand such that all four detecting templates are available for identification during the next round in the sequence;

FIG. 5 depicts an example array where each well of the reactor area is in fluidic connection with a dedicated detection area and all four nucleotide recognition elements 1 are flowed through a flow chamber from a reagent preparation area 13 to a collection area 18 and simultaneously allowed to react with the next in sequence element in the growing structure whereas all non-complementary recognition elements are moved via electric potential or other fluid motive force to the dedicated detecting areas for identification of the next element in sequence in each individual sequencing template.

### DETAILED DESCRIPTION OF THE PREFERRED EMBODIMENTS

The present invention relates to a process of identifying individual units in a self-assembling number of elements as they are assembled into a structure. Without limitation, the instant invention is more specifically directed toward sequencing of a deoxyribonucleotide structure into its individual monomer units. Thus, the present invention has utility as a DNA sequencing process and apparatus.

Generally, determining the sequence of a structure containing N different elements requires a system having N sets of chambers. Each of N sets in turn contains N chambers. To initiate the assembly process a different element is placed in each chamber in every set. For example the i-th chamber in every set contains the i-th element. It is appreciated that each element is optionally affixed to a wall or structure or support or to the bottom of a chamber. When the target sequence is unique, i.e. the next element is always different from the previous one, the next step in the process is to put the same element into each chamber of the same set but different elements across sets. That is, all chambers of the i-th set will receive i-th element. Since there are N sets each set containing N chambers, there are N×N possible two element strings. There will be one and only one chamber where a second element will bind to the first element to begin growing the structure. In all other chambers there will be no addition of one element to the other. Thus, by subtraction it is identified what the sequence of the first and second elements are. For example, if the growth of the structure happened in the i-th chamber of the j-th set then the first element in a sequence is the i-th and the second one is the j-th where i and j are integers greater than zero and less than or equal to N.

All sets not containing the chamber wherein a two element structure was formed are discarded such that only one set of N chambers remains. It is appreciated that all chambers are then optionally washed of free unbound elements so that the only remaining structure in the system is the surface bound or solution two element structure. At this point an excess amount of the identified element is added to each chamber of the i-th set so that all chambers contain the same two element structure.

Finding the third and every other element in the sequence is reduced to a simple algorithm. N different elements are added into N chambers to reveal the next element. All chambers are then optionally washed of the elements from all N chambers and the identified element is added to all chambers in excess to grow the structure one more unit. By repeating the steps for every member of the unidentified sequence the element order is easily determined.

In a nonlimiting example, an unidentified DNA template sequence is resolved by the instant inventive process. DNA is comprised of four element types, an adenine, guanine, thiamine, and cytosine. Therefore, the integer N is equal to 4. It is appreciated that DNA is optionally synthesized in vitro in a chamber in the presence of all required molecules illustratively including a DNA polymerase and a helicase. It is further appreciated that with a given sequence of DNA only one of the four types of elements A, T, G or C will be assembled in with the target sequence at each hybridization site being identified. It is known in the art that A hybridizes to T and G hybridizes to C. If the next element in an unknown sequence of DNA is a T only the chamber containing an A recognition element will produce extension, thus, removing the A element from solution. All other chambers will contain free nucleotide. By identifying which wells contain free nucleotide the sequence of the target is deciphered. Thus, according to the inventive process DNA sequencing is illustratively performed using four chambers with the appropriate number of DNA molecule copies in each chamber.

Of primary importance to the inventive process is that each step is conceptually split into many small steps. Each small step consists of supplying one dose of elements wherein the number of DNA template copies is much larger than the number of elements delivered during each small step. Therefore, if a particular element is incorporated into a DNA molecule at the next unoccupied site in the sequence, it is appreciated that the number of free monomers is negligible in solution after the first small step. This process allows simple identification of repeat elements (or copy number of a particular template) in the structure sequence. In a nonlimiting example the total number of DNA copies of template copies in the chamber is ten times larger than the number of elements in each single dose. It is appreciated that the first dose of elements being one-tenth that of the number of DNA will occupy sites on one-tenth of the DNA molecules leaving nine-tenths of the sites on the DNA template molecules free. After ten small additions of elements all sites on the template DNA will be occupied. Between and after each addition one-tenth concentration of element it is appreciated that there will be no free monomer elements in solution because all of the elements will be incorporated into DNA molecules. Upon addition 11 observation of free monomers in the solution occurs which signals completion of the current step and beginning of the next one. As such, a primary advantage of the instant invention over the prior art is a rapid and accurate process for revealing repeat elements or nucleotides in the sequence.

In a nonlimiting example the chamber under consideration contains 100,000 copies of DNA template molecules. One dose of monomer elements contains 10,000 molecules of monomer. Therefore, it will require 10 doses of monomer element to fill the vacancies in all copies of DNA molecules. The eleventh dose will create 10,000 monomer elements available for detection in the solution signaling that the site is not a repeat and, further, signaling the next recognition step.

During sequence identification only one chamber out of four will incorporate elements into the growing DNA structure the other three chambers will demonstrate free monomers in solution, thus, revealing the nature of the monomer which is incorporated into the growing DNA structure. After identification of which monomer element is incorporated at the particular site sufficient copies of that identified monomer element is optionally added to the other three chambers, thus, occupying that site on a growing DNA structure in all four chambers. That completes the final step of the process and determination of the entire sequence of the unknown DNA template molecule is similarly determined.

The elements supplied to the chamber are classified by purpose. The first type of element is a recognizing element. Recognizing elements are elements identifiable as free in solution after being unable to bind in a complementary fashion to the DNA template at the next available site. The second type of element is a building element. Building elements are elements not intended to be used for recognition, however, it is appreciated that they are optionally used as recognizing elements. Building elements are designed to occupy free sites in all growing DNA structures left vacant by the non-complementary recognizing elements in a chamber.

The inventive process is operable for many different types of sequence structures or element containing structures. A recognition element or a building element is illustratively a nucleotide, a ribonucleotide, deoxyribonucleotide, deoxynucleotide, peptide nucleotides, modified nucleotides, modified peptide nucleotides, modified phosphate sugar backbone nucleotides, amino acids, or modified amino acids. Although chemically similar, a recognition element is mainly involved in an inventive process for nucleotide identification or sequencing while a building element is applied for a collateral determination of copy number of a particular DNA template.

Recognition elements are optionally labeled. A single label or multiple labels are optionally present on each individual recognition element. In a nonlimiting example, during DNA sequencing four different types of recognition elements are employed: A, T, G, or C. Each recognition element optionally contains the same label or different labels that are distinguishable from each other based on characteristics of the combination of label and the remainder of the recognition element. Labels are optionally bound to one or multiple sites on a recognition element such as in a nucleotide. Recognition elements optionally have a label attached at a base, on a sugar moiety, on the alpha phosphate, beta phosphate, gamma phosphate, or any combination thereof. Illustratively, a label for adenine preferably has a fluorophore bound to the gamma phosphate wherein the fluorophore is distinguishable from a fluorophore bound to the gamma phosphate on a different species of recognition element. Thus, in the case of DNA sequence the four recognition element species optionally contain four different fluorophores.

Multiple label types are operable in the instant invention illustratively including chromophores, fluorescent moieties, enzymes, antigens, dyes, phosphorescent groups, chemiluminescent moieties, scattering or fluorescent nanoparticles, Raman signal generating moieties, fluorescence resonance energy transfer donor or acceptor molecules, precursors thereof, cleavage products thereof, and combinations thereof. In addition, it is appreciated that the label on any recognition element is optionally photo bleachable, photo quenchable, or inactivatable. A recognition element is optionally bound into a single strand of growing DNA in the formation of a structure, and prior to, during, or subsequent to the addition of this recognition element the label is photo bleached such that contamination of the fluorescence of the label does not interfere with subsequent identification steps.

Identifying the presence or absence of free recognition elements in a chamber is dependent on the type of label present on the individual recognition elements. Numerous identifying methods are known in the art illustratively including far field microscopy, near field microscopy, evanescent wave or wave guided illumination, nanostructure enhancement, mass spectroscopy, photon excitation, multi photon excitation, FRET, photo conversion, spectral wavelength discrimination, fluorophore identification, background suppression, electrophoresis, surface plasma in resonance, enzyme reaction, fluorescence lifetime determination, radio frequency modulation, pulsed multiline excitation, or combinations thereof.

It is appreciated that the structures are optionally complementary to a template structure that guides which element is placed in the next location in the sequence. Illustratively, the template structure is a DNA oligonucleotide sequence. Template DNA sequences are optionally free in solution or bound to a support in a reaction chamber or to the reaction chamber wall itself. Immobilization of the template is accomplished through conventional techniques known in the art illustratively including covalent attachment to a functional group on the solid surface, or by biotin/avidin interaction. In an optional embodiment a short oligonucleotide primer is bound to a support. The oligonucleotide segment is complementary to a small known sequence on the DNA template strand. Hybridization of the DNA template strand with the surface bound oligonucleotide immobilizes the DNA template to the surface of the chamber in reversible fashion. This embodiment has the additional advantage of providing a primer sequence for a polymerization reaction to occur. It is common in the art of DNA sequencing analyses that small segments of known sequence are present at the termination of each unknown strand. The template strand is optionally double stranded DNA, single stranded DNA, single stranded DNA hairpins, RNA, or RNA hairpins.

The inventive process further comprises a polymerization reaction in which one unknown recognition element or building element is added to the growing DNA structure in a complementary fashion. The polymerization reaction is performed by a nucleic acid polymerizing enzyme that is illustratively a DNA polymerase, RNA polymerase, reverse transcriptase, or mixtures thereof. It is further appreciated that accessory proteins or molecules are present to form the replication machinery. In a preferred embodiment the polymerizing enzyme is a thermostable polymerase or thermodegradable polymerase. Use of thermostable polymerases is well known in the art such as Taq polymerase available from Invitrogen Corporation. Thermostable polymerases allow a recognition or building reaction to be initiated or shut down by a change in temperature or other condition in the chamber without destroying activity of the polymerase.

Accuracy of the base pairing in the preferred embodiment of DNA sequencing is provided by the specificity of the enzyme. Error rates for Taq polymerase tend to be false base incorporation of 10⁻⁵ or less. Johnson, Annual Reviews of Biochemistry, 1993: 62:685-713; Kunkel, Journal of Biological Chemistry, 1992; 267:18251-18254 (both of which are hereby incorporated by reference.) Specific examples of thermostable polymerases illustratively include those isolated from Thermus aquaticus, Thermus thermophilus, Pyrococcus woesei, Pyrococcus furiosus, Thermococcus litoralis and Thermotoga maritima. Thermodegradable polymerases illustratively include E. coli DNA polymerase, the Klenow fragment of E. coli DNA polymerase, T4 DNA polymerase, T7 DNA polymerase and other examples known in the art. It is recognized in the art that other polymerizing enzymes are similarly suitable illustratively including E. coli, T7, T3, SP6 RNA polymerases and AMV, M-MLV, and HIV reverse transcriptases.

The polymerases are optionally bound to a primer template sequence. When the template sequence is a single-stranded DNA molecule the polymerase is bound at the primed end of the single-stranded nucleic acid at an origin of replication or with double stranded DNA to a nick or gap. Similarly, secondary structures such as in a DNA hairpin or an RNA hairpin allow priming to occur and replication to begin A binding site for a suitable polymerase is optionally created by an accessory protein or by any primed single-stranded nucleic acid.

In a preferred embodiment the template is bound to a support located within the chamber. Materials suitable for forming a support optionally include glass, glass with surface modifications, silicon, metals, semiconductors, high refractive index dielectrics, crystals, gels and polymers. A support is illustratively a planar or spherical surface. It is appreciated in the inventive process that either a sequencing primer in the case of DNA sequencing, a target nucleic acid molecule, or the nucleic acid polymerizing enzyme are illustratively immobilized on the support. A complementary bonding partner for forming interactions with any of the above molecules or any other of the operational machinery in the inventive process are similarly appreciated to be suitable for immobilizing material onto a surface. Interaction of any of the replication machinery with the surface is optionally nonspecific. Examples of a specific type bonding interaction include a biotin/streptavidin linkage wherein a known primer sequence is optionally labeled with a biotin and the solid support is labeled with a streptavidin. When the biotin primer is added to the chamber a tight bonding interaction between the biotin and streptavidin occurs immobilizing the primer sequence onto the support surface. It is further appreciated that the target DNA sequence is optionally labeled itself so that it is immobilized on the support surface. Additionally, a primer sequence is optionally immobilized by hybridization with a complementary immobilized oligonucleotide. Thus a primary oligonucleotide is immobilized on a surface with a short sequence complementary to the primer oligonucleotide. It is preferred that the primer oligonucleotide is of sufficient additional length that hybridization between the immobilized nucleotide and the primer oligonucleotide allows base pairing between the primer oligonucleotide and the target DNA sequence, thus, binding the target DNA sequence to the support surface. Interaction of any suitable molecule to the support surface is appreciated to be reversible or irreversible. Alternative exemplary methods for immobilizing sequencing primer or target nucleic acid molecule to a support include antibody antigen binding pairs or photoactivated coupling molecules. It is appreciated in the art that numerous other immobilizing methods are similarly suitable in the inventive process.

It is further appreciated that the proteinaceous material of the polymerization enzyme in the case of a DNA polymerase is optionally immobilized on the surface either reversibly or irreversibly. For example, RNA polymerase was successfully immobilized on activated surface without loss of catalytic activity. Yin et al., Science, 1995; 270: 1653-57, which is hereby incorporated by reference. Alternatively, an antibody antigen pair is utilized to bind a polymerase enzyme to a support surface whereby the support surface is coated with an antibody that recognizes an epitope on the protein antigen. When the antigen is introduced into the reaction chamber it is reversibly bound to the antibody and immobilized on the support surface. A lack of interference with catalytic activity in such a method has been reported for HIV reverse transcriptase. Lennerstrand, Analytical Biochemistry, 1996; 235:141-152, which is hereby incorporated by reference. Additionally, DNA polymerase immobilization has been reported as a functional immobilization method in Korlach et al., U.S. Pat. No. 7,033,764 B2; incorporated herein by reference. Finally, any protein component can be biotinylated such that a biotin streptavidin interaction is optionally created between the support surface and the target immobilized antigen.

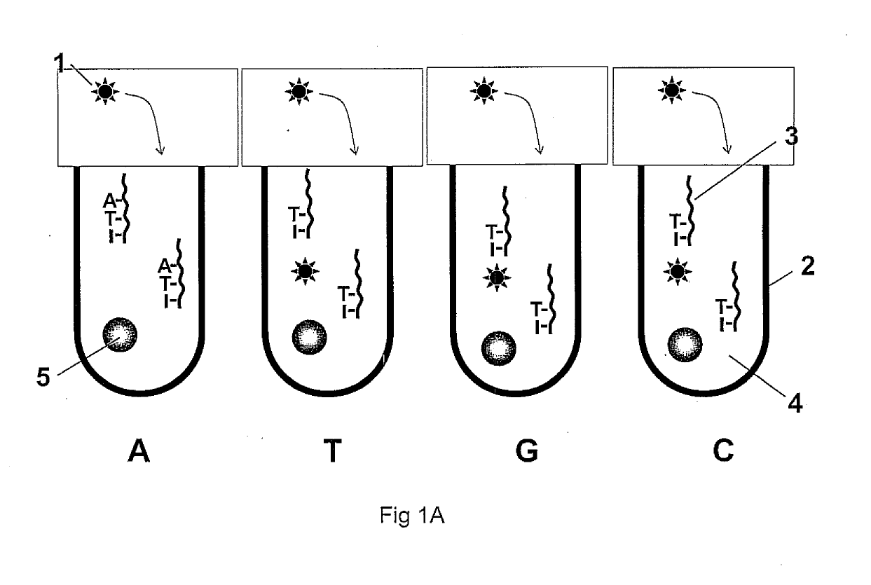

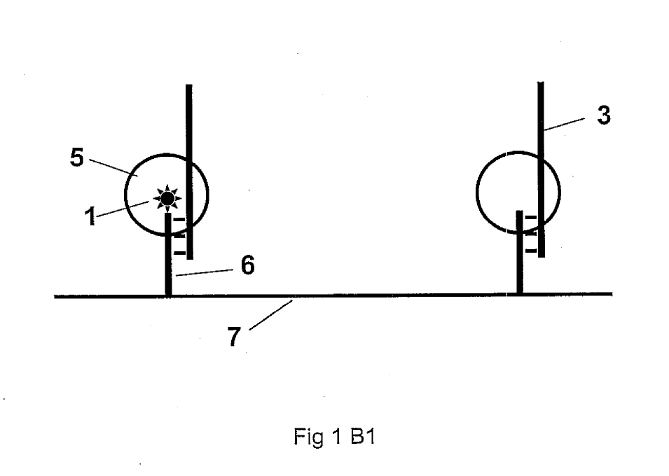

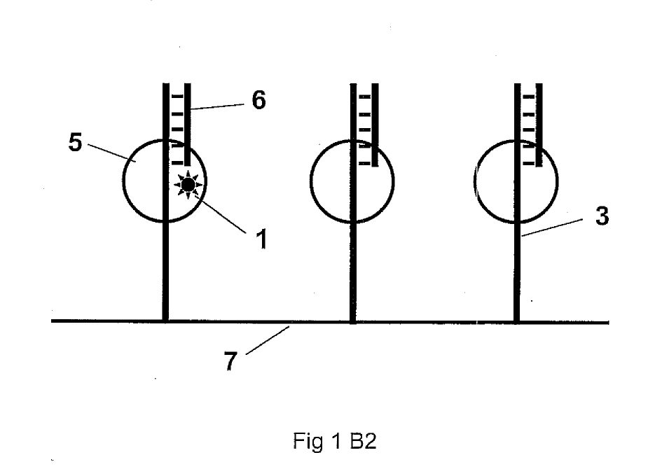

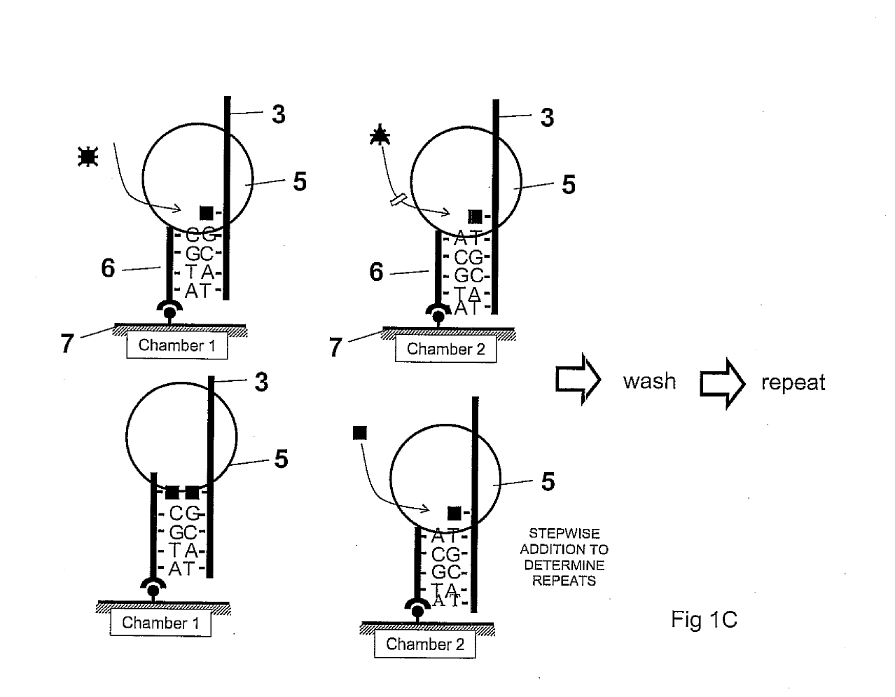

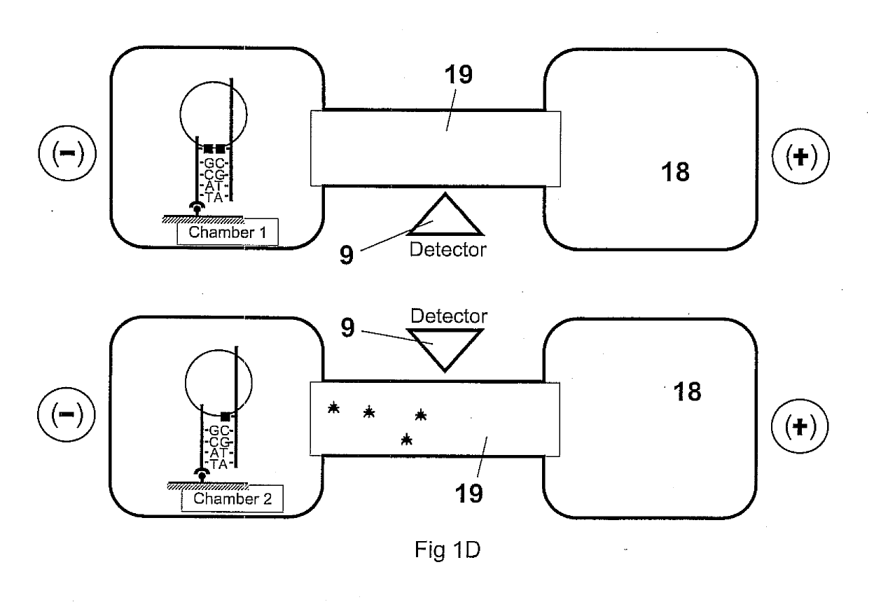

In a preferred embodiment both the target and the polymerase remain free in solution. Referring to FIG. 1, the sequencing procedure is initiated in a solution optionally containing the DNA template 3 as well as one of N species of recognition elements 1. Illustratively, four recognition element species are available represented by A, T, & and C. The four reaction chambers correspond to each species of recognition element wherein an individual species of recognition element is added. In each chamber a reaction is optionally initiated by the addition of a nucleic acid polymerizing enzyme. In an alternative embodiment a primed target sequence may be established by pre-addition of a target sequence, a primer, and a species of recognition element. No structure extension occurs in the absence of a DNA polymerase. The reaction is initiated by addition of the DNA polymerase. In an alternative embodiment all components of the replication machinery are present including the DNA polymerase, the template molecule, the primer, and a particular recognition element. The solution in this embodiment is optionally void of necessary ions for the function of the polymerase enzyme. For example, the reaction may be initiated by the addition of magnesium ions such that the replication machinery now becomes functional. In yet another alternative embodiment all of the reaction machinery is present, however, the reaction chamber is heated above a threshold temperature above the melting temperature of the template molecule and the primer such that hybridization between the primer and the template molecule does not occur. The polymerization reaction begins by adjusting the temperature to a suitable reaction temperature.

In the preferred embodiment depicted in FIG. 1A recognition element species are added to each chamber to initiate a polymerization reaction. Recognition elements are thereby diffused through the fluid medium or forced to flow through the chamber via hydrodynamic pump rapidly bringing the recognition element into association with the polymerization machinery. A recognition element inserts into the active site of the polymerase and the polymerase establishes whether this nucleotide analog is complementary to the first open base of the target nucleic acid molecule or whether a mismatch has occurred. In the reaction chamber illustrated in FIG. 1A where an A recognition element is added to the reaction chamber it is appreciated that the template at the next recognition site is defined by a T. Should an A recognition element be incorporated into the polymerase a positive match will occur and the polymerization machinery will form a covalent bond between the A and the primer sequence. However, in the second tube where a T recognition element is added a mismatch occurs and no polymerization process will proceed. If the ratio between the recognition element and the template is proper such that the recognition element illustratively is one-tenth the concentration of the template it is appreciated that all of recognition element in the A chamber will be bound and polymerized at the first hybridization site on the template molecule.

Each chamber is optionally in fluidic connection with a detector such that by washing each of the chambers free recognition elements are transported to the detector area and are readily detected. In a preferred embodiment each of the recognition elements is differentially labeled such that it can be easily distinguished from other recognition elements. Thus, a single detector is employed whereby the individual unincorporated element species are readily identified, thus, determining the sequence at the first hybridization site in the template molecule.

As depicted in FIG. 1D, an electrophoretic gel such as that formed by acrylamide, agarose, or other material known in the art is used intermediate each reaction chamber and a dedicated collection area 18 with a detector intermediate therebetween. Following sufficient time for all recognition elements to hybridize with the template strand, an electric potential is applied moving the free nucleotides past a detector 9 to the collection area. After identification of the next element in sequence, all unused elements are optionally returned to their respective recognition chambers by reversing the polarity of the electric field. This embodiment of the invention has the advantages of reducing reagent costs and time between sequencing iterations while simultaneously providing a reversible washing step for improved sequence addition.

Alternatively, numerous collection chambers are optionally employed. In a nonlimiting example each reaction chamber serves the additional roles of repeat detection chamber and sequence building chamber. Following addition of recognition elements to the recognition chambers, a first electric potential is applied to move all free recognition elements past a detector to identify the next element in sequence. This removes all unbound recognition elements from the reaction chambers. A portion or all of the reaction chambers are then used as repeat detection chambers whereby additional recognition elements are optionally added to determine the repeat number if any. A second electric potential is then applied to remove all unbound elements from the recognition chambers to a second set of collection areas. The first electric potential is then applied with reverse polarity to move all the unhybridized recognition elements back into their original recognition chambers negating the need to add more recognition elements to the N−1 chambers that did not demonstrate hybridization, thus, saving reagent and expense.

In an alternative embodiment a sampling of each of the reaction chambers or the repeat detecting chamber is obtained and injected into a mass spectrometer to recognize the presence of free elements. This embodiment has the advantage of using native, non-labeled elements whereby greater efficiency and accuracy of the polymerase is achieved. Alternatively, it is appreciated that multiple detector types are optionally employed. In a nonlimiting example, the recognition elements are fluorescently labeled. Detection of the species of hybridizing recognition element is, thus, detected by a fluorometer. After washing all unbound recognition elements from the reaction chambers, repeat detection is accomplished by addition of unlabeled recognition elements or building elements. Free elements are optionally detected by a mass spectrometer. This has the advantage of allowing washing of the chamber and use of N chambers for recognition, repeat detection, and building. Also, use of unlabeled elements in the repeat detection phase allows N replicates of repeat detection without contamination of the next round of sequence recognition.

In a preferred embodiment the template is bound to a support. Washing of the chambers removes only unbound recognition element. In an alternative embodiment the fluidic connection between each reaction chamber in the detector is such that the large template molecule remains in the chamber while the small recognition elements are readily transported through a barrier such as a size exclusion membrane or an electrophoretic gel. As such, each chamber is washed free of unbound recognition elements.

Once the complementary species of the recognition element is identified, this recognition element species is optionally stepwise added to each of the four chambers. In a nonlimiting example one-tenth concentration of recognition element was initially added to each of the four chambers for identification purposes. Stepwise addition of one-tenth concentration recognition elements allows for gradual saturation of all hybridization sites on each of the template strands. Should the stepwise additions exceed a value of ten it is understood that there is a repeat. For example if 20 stepwise additions are required before saturation of all hybridization sites on the template molecule occurs, it is appreciated that there is a single repeat on the template strand.

After identification of the particular recognition element species bound to the template molecule and determination of whether or not a repeat of that particular recognition element species occurs, building elements are added to each of the chambers at a known concentration to fully saturate all structures. At this point all unbound recognition element and building element species are optionally washed from each chamber and the reaction cycle begins again so as to determine the next recognition element species in each of the template strands. Thus, by repeating the sequence of steps the sequencing primer is extended and the entire sequence of the target is determined.

It is appreciated that the solution be of suitable extension medium so as to permit diffusion, incorporation, and washing out of each of the reaction chambers. In a nonlimiting example suitable extension media a contains 50 mM Tris-HCl pH 8.0, 25 mM magnesium chloride, 65 mM sodium chloride, 3 mM DIT, and elements at appropriate concentration to permit identification of the sequence. It is appreciated that other extension medium are similarly suitable and optimized for the particular polymerase or template being utilized.

In an alternative embodiment as in the present nonlimiting illustration, a fifth chamber is present termed a repeat detecting chamber wherein, following identification of the recognition element species, recognition element species is added to the repeat detecting chamber to determine whether or not a repeat exists and the number of repeats in sequence. Following identification of both the recognition element species in the original reaction chambers as well as the number of repeats in the repeat detecting chamber, a suitable concentration of building elements is added to all chambers to fully saturate all sites at that portion in the growing structure. It is appreciated that a washing out procedure is optionally employed between each subsequent sequence whereby the unbound elements in all reaction chambers are removed.

In an alternative embodiment the repeat detecting chamber is in fluidic communication with each of the four reaction chambers. All the reaction solution from each of the four reaction chambers is transferred to the repeat detecting chamber. It is in the repeat detecting chamber that stepwise addition of the identified species of recognition element is added to determine whether or not a repeat exists. Once the presence of a repeat is determined, or shown not to exist, the repeat detecting chamber is optionally washed free of all unbound recognition elements and the fully hybridized growing DNA molecule is subsequently transferred back to each of the four reaction chambers for the next round of element recognition.

In an alternative embodiment there is no washing out of the elements which are left in solution as excessive free elements after each of the previous steps. However, it is appreciated that the ratio between recognition elements and template is such that there is little to no observable contamination as the procedure moves through several rounds of recognition. For example, in a situation with five chambers, four recognition chambers and a repeat detecting chamber, four contain copies of free DNA molecules to be sequenced. Each chamber is initially supplied only with a small dose of one species of recognition element. This small dose is illustratively one-tenth concentration of target DNA molecules to be sequenced. After identification of the next hybridizing recognition element that element is added to the fifth chamber only using small doses to determine if there is a repeat. After the correct dose of that element is determined, the appropriate concentration of building element is added to the first four chambers and the next round of recognition begins.

In yet another alternative embodiment a sixth chamber is present termed a sequence construction chamber. Four recognition chambers contain copies of free DNA molecules to be sequenced, and each chamber is supplied with only a single species of recognition element. After the next hybridizing element is identified as described in the previous procedures, that element is added to the fifth repeat detecting chamber to determine if there is a repeat. Subsequently, the solution from all five chambers is then moved to a sixth chamber where the appropriate number of building elements for all six chambers is added to the resulting solution so as to fully saturate all free sites in the growing structure that are complementary to the current hybridization site on the template. Following saturation of all sites the solution from the sequence construction chamber transferred back to each of the four recognition chambers and the repeat detecting chamber. All chambers now contain a DNA template molecule hybridized to a growing structure of equal length and a new round of recognition element species identification occurs.

In an alternative embodiment, after identification of the next complementary recognition element each of the four recognition chambers is emptied and washed such that the solution from each of the four chambers is fully discarded and not sent to the fifth or sixth chamber. The correct amount of the determined element is added to the fifth chamber, which is a large chamber, so as to fully saturate all hybridization sites on the template DNA molecule in this chamber. Small volumes of the fifth repeat detection chamber are subsequently added back to each of the recognition chambers for a new round of recognition species identification. Optionally, a washing out procedure occurs during transfer of solution from each of the recognition chambers to a fifth repeat detecting chamber.

In an alternative process employing six chambers, five small chambers and one large sequence construction chamber, five small volumes of target DNA molecules in solution, or immobilized on a support in suspension, are transferred from the sequence construction chamber to each of the five other chambers. The next element in sequence is determined as described above. The fifth chamber is then used to determine the number of possible repeats. After the recognition element species is identified and the number of repeats is determined, the solution from all five chambers is transferred back to the sixth chamber where a correct amount of the determined element is added and the whole procedure is then repeated. It is appreciated that in this embodiment simultaneous sequencing and amplification of target DNA occurs. For example, in the situation where one large sixth sequence construction chamber is utilized small samples are withdrawn and divided amongst the four recognition chambers and the fifth repeat detecting chamber. The next element in series is identified and the presence of repeats is determined. The proper dose of building elements is then added to the sequence construction chamber to fully saturate all sites on the growing DNA structure.

In a preferred embodiment, recognition of the next element is sequence is accomplished by detecting growth of a complementary strand to a detection template. A detection template is illustratively a homopolymer. In a nonlimiting example, a detection template is a 100-mer cytosine, guanine, thymine, or adenine. It is appreciated that shorter or longer oligomers are similarly operable. The detection template is optionally free in solution or selectively immobilized on a surface in a recognition chamber.

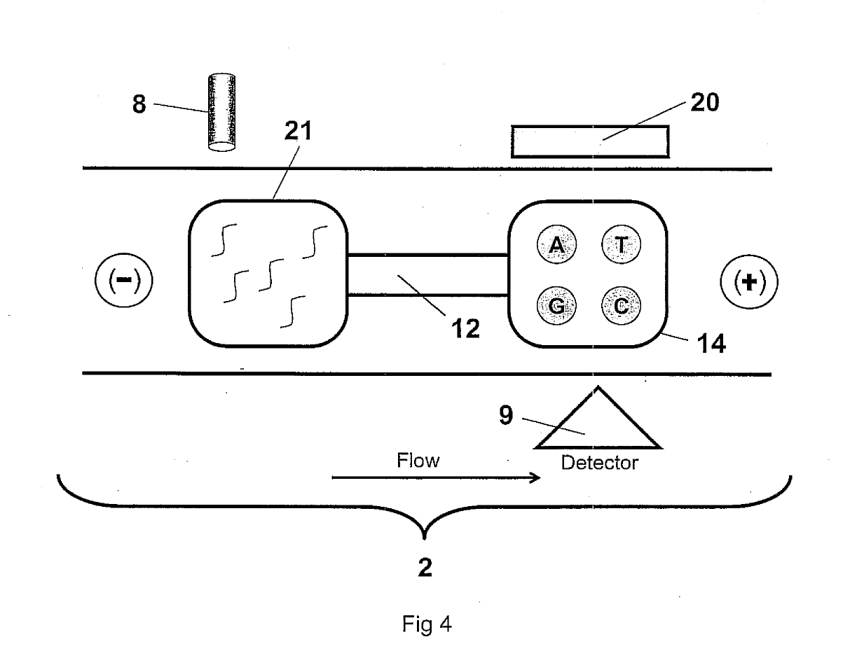

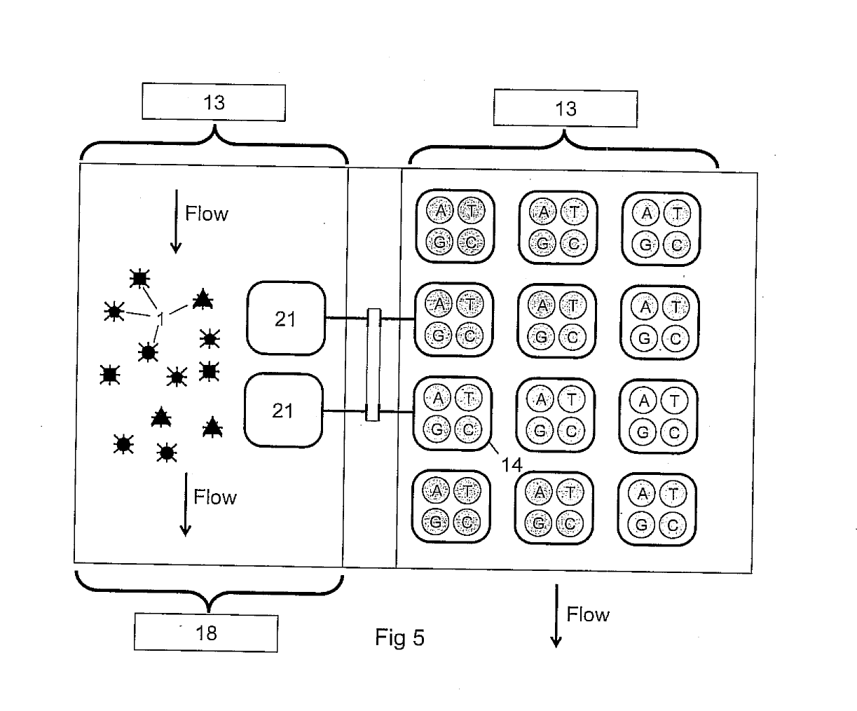

As depicted in FIGS. 4-5, a recognition chamber is optionally comprised of a reactor area and a detector area. In a preferred embodiment an unknown sequencing template is immobilized in a reactor area. Four detection templates are similarly immobilized in a detector area in fluidic connection with the reactor area. Each reaction chamber also contains materials necessary for DNA replication illustratively including a polymerizing enzyme and cofactor molecules. A single or series of microdispensers adds a species or multiple species of recognition elements to the reactor area. If the recognition element is complementary to the next element in sequence on the sequencing template, the polymerization enzyme incorporates that element into the growing structure, hence, removing all recognition element from the solution in the reactor area. If the recognition element is not complementary to the next element in sequence the element remains in solution.

An electric field is illustratively applied to move all unbound recognition elements from the reactor area to the detector area where detecting template is immobilized. However, it is appreciated that other mobilization methods are similarly operable illustratively including a mechanical flow system, a pressure system, capillary action, diffusion, holographic pump, or other suitable method known in the art. As all components for polymerization are also present in the detection area, all free recognition elements are free to form a fully assembled DNA molecule on the detecting templates that are comprised of complementary elements.

In a preferred embodiment, each of four nucleotide elements are individually labeled illustratively by Cy5, Cy3, Texas Red, or FITC such that each species of recognition element is individually recognizable by a fluorescent signature. The detection area is optionally comprised of four sub areas each containing a homogenous population of detecting templates comprised of a homogenous nucleotide species. Illustratively referring to FIGS. 4-5, area A has a polyadenine, area B has a polyguanine, area C has a polycytocine, and area D has a polythymine. It is appreciated that a detecting template is optionally defined by a heterogeneous species of nucleotide. Following sufficient incubation time of the recognition elements with the sequencing template, any free recognition elements are transported to the detection area where full primer extension occurs on the detecting template complementary to the recognition element producing a long chain double stranded DNA molecule (dsDNA) in the detection chamber. An optional wash procedure is applied to remove any unbound recognition element and the position on the detection area array wherein double stranded DNA is present is readily detected by fluorescence.

This preferred embodiment has numerous advantages including using low levels of recognition element in each identification sequence. This is bolstered by the observation that a complementary structure comprised of a long polymer of labeled recognition element produces a strong fluorescence signal. Further, the dsDNA structures synthesized in the detection area are confined to a small area further concentrating the signal and allowing for a small detection array.

It is appreciated that any method of detecting double stranded DNA is similarly operable in the instant inventive process. Examples illustratively include using reversible intercalating agents such as ethidium bromide, doxorubicin, thalidomide, isopropyl-oxazolopyridocarbazole, or 9-aminoacridine. Additional examples illustratively include mass spectroscopy, specialized gel pores, surface plasmon resonance, atomic force microscopy, electrophoresis, migration, or antibody interaction. It is appreciated that other methods of capture or identification of double stranded DNA are similarly suitable in the instant invention.

Following identification of the next element in sequence, the detection array is optionally regenerated by a wash step wherein the double stranded detecting DNA is melted separating the two strands. In a non-limiting example, a 100-mer polyA detecting template is melted from a polyT structure strand by heating the detector area array to 66.6° C. For a 100-mer polyG detecting template, the polyC structure stand is optionally melted at a temperature of 86.6° C. It is appreciated that other melting temperatures are operable and are chosen based on the length and composition of the detecting template sequence. Application of flow or an electrical field moves the non-immobilized strand from the detection area leaving only immobilized single stranded DNA available for a subsequent round of identification. The identification procedure is optionally repeated for another species of recognition element until the entire structure is completed and the sequence identified.

In a preferred embodiment a recognition chamber is an array with a large number of reactor areas each fluidically connected to a detection area dedicated to its respective reactor area. A high density array plate is illustratively manufactured from transverse slicing of a fiber optic block. An example of this process is described in Margulies, M., et al., Nature, 2005; 437:376-80, the contents of which are incorporated herein by reference. The instant inventive process is achieved by fluidically connecting an array block that represents the reactor area to a micro detection area wherein 1-4 homopolymeric detecting templates are immobilized. Example systems and methods for microfluidic connection are achieved by multilayer soft lithography similar to that developed by Fluidigm Corp. (San Francisco, Calif.), or Labchips developed by Caliper Life Sciences (Mountain View, Calif.). In this embodiment an array of microfluidically connected channels allows for rapid, high throughput detection of hundreds of unknown sequences simultaneously.

Repeat detection is optionally performed in a repeat detecting chamber or in the recognition chamber itself. Optionally, the copy number of recognition element added to the reactor area of the recognition chamber is sufficiently low as to not fully saturate all available hybridization sites on the sequencing templates. By a similar iterative process to that described for a repeat detecting chamber, recognition element is added in a stepwise fashion until recognition element of that species is detectable in the detection area. Simple calculation identifies the number of repeats on the sequencing template.

In a most preferred embodiment, four recognition elements are simultaneously added to all wells of a sequencing array of reactor areas. Each of the recognition elements is differentially labeled and reversibly terminated. It is appreciated that the termination and label are optionally the same component or multiple components on the same recognition element. The reactor area and detection area of the recognition chamber contain immobilized sequencing template and detecting template, respectively, along with DNA polymerase and necessary buffer and cofactor reagents for a polymerization reaction. The copy number of the sequencing template is illustratively 100× that of the copy number of each species of homopolymeric detecting template and the detecting template is illustratively between a 25-mer and a 50-mer. The density of sequencing template is such that only a single species of sequencing template is present in each well of the array. Methods of limiting and expanding a genomic library suitable for use in the instant invention are described in Margulies et al., 2005.

All species of recognition elements are added to all wells of the array simultaneously. As each species is reversibly blocked, only the next in sequence species will be incorporated into the growing structure at the next hybridization site in any individual well. Thousands of different sequences are simultaneously subjected to identification of the next element in the respective sequence. The unhybridized recognition elements are then fluidically transferred to each well's detection area, are deprotected, and subjected to a polymerization reaction on all detector templates simultaneously. Identification of the next element in sequence in the sequencing chamber is achieved by determining which detection template is not used as a template for a polymerization reaction. Illustratively, if a particular reactor area holds a sequencing template species with T as the next element in sequence, A recognition elements will be incorporated into the structure and will not be transferred to the detection area. Upon transfer and deprotection, the T, G, and C recognition elements are free to form double stranded DNA on their respective detection templates, whereas the T-detecting template does not have a second strand added due to depletion of the A recognition elements in the reactor area. The detection area is optionally washed and a fluorescent detector identifies which element was incorporated into the sequencing template. Following identification of the next element in sequence, the detection area is heated to melt the double stranded DNA, the area is washed and fresh polymerase is added regenerating the detecting area for a subsequent round of identification.

Each well in the array optionally has a different sequence of sequencing template. By spatially isolating each signal thousands of unknown sequences are simultaneously determined. Recognition elements in the detection area are optionally deprotected at the same time the elements incorporated into the next site on the sequencing template are also deprotected and washed so that a fresh round of recognition elements are added identifying the next in sequence.

Repeat recognition is readily achieved. As each recognition element is protected, a second addition of that element is not placed in the next hybridization site on the sequencing template. As all recognition elements are moved from the reactor area prior to deprotection, no free recognition elements are available to add to the sequence until a new round of identification is initiated. Thus, a repeat region is identified in a stepwise fashion similar to any other element in sequence.

By careful selection of the ratio and length of sequencing template to detecting template, as well as recognition element to sequencing template, all sites on the sequencing template are illustratively filled in each round of element recognition while simultaneously providing sufficient recognition element to readily identify the incorporated species. Each recognition element incorporated into the hybridization site is optionally deprotected and the fluorophore cleaved producing a native nucleotide element in the growing structure enhancing the activity of the polymerase and reducing error.

It is appreciated that numerous other embodiments of the instant invention exist with greater or fewer chamber numbers, types, sizes, interconnections, or pathways and are also the subject of the instant invention.

An embodiment of the instant invention includes an apparatus. This apparatus optionally employs numerous reactor types illustratively including a batch reactor, a plug flow reactor, or a drop reactor. An apparatus for self-assembly of a number of elements comprises a reaction area that contains a suitable number of chambers relative to the number of different species of elements in the growing structure; a preparation area in fluidic connection with the reaction area whereby reagents and solutions are prepared to be delivered to the reaction area in stepwise or simultaneous fashion; and a detection area in fluidic, physical, or optical connection with the reaction area.

The detection area employs any suitable detector for detection of the type of label on each of the individual recognition elements. For example, if each of the recognition elements is labeled with a particular fluorophore a fluorescent detector is employed so as to identify which chambers contain free recognition elements. In the case where either unlabeled recognition elements are employed or nonoptically resolvable recognition elements are employed each of the reaction chambers is illustratively connected to a mass spectrometer whereby the presence of free recognition elements is readily determined.

In the inventive apparatus the reaction area has N recognition chambers, each chamber having a plurality of microdispensers. The number of microdispensers is related to the number of possible recognition element species. For example, if there are four recognition element species each chamber in the reaction area has four microdispensers to allow distribution of the various species of recognition element. In an alternative embodiment there are eight microdispensers aimed at each of the reaction chambers such that any of the four recognition elements are optionally distributed to each reaction chamber as well as any building elements without fear of contamination between the elements. Thus, each microdispenser is filled with one type of element so that each type of element is available to be distributed into each chamber in the reaction area. In the case of five small chambers the fifth chamber similarly has four or eight microdispensers for delivery of elements to that chamber. It is appreciated that the number of microdispensers is optionally related to the number of the elements in the growing structure. In the case of ten separate element species as many as ten or twenty microdispensers for each chamber are employed. Alternatively, a single or fewer than N microdispensers is employed with a washing out step of each of the microdispensers between delivery of different recognition or building elements.

In a preferred embodiment, an apparatus for performing sequence identification by synthesis and detection of a parallel homopolymeric detecting template is achieved by illustratively administering to a reaction chamber four sets of liquids. The first provides a solution containing as one of the active components one type of monomer. In a preferred embodiment the monomer is a single species of nucleotide. The second liquid provides solution with one of the active components being a polymerizing enzyme—illustratively a DNA polymerase. The third liquid illustratively contains a nucleic acid template such as a sequencing template that optionally has a portion of known sequence hybridized to a primer to form a short double stranded DNA region that can be sequentially extended by addition of complementary nucleotides to the primer. The fourth liquid illustratively contains as one active ingredient detecting template. The sequencing and detecting templates are optionally free in solution or immobilized on the surface of a small carrier such as a micro-particle. Examples of micro-particles illustratively include polystyrene spheres and streptavidin coated paramagnetic beads optionally generated and as described by Shendure, J, et al., Science, 2005; 309:1728-32, the contents of which are incorporated herein by reference. It is appreciated that other surfaces known in the art are similarly operable.

The recognition chamber is illustratively divided into a reactor area and a detection area. The sequencing template is optionally immobilized in the reactor area by adhesion to a surface. The reaction area illustratively contains a buffer solution, an oil emulsion, or an acrylamide or agarose based gel system wherein the sequencing template is deposited.

The device has a means of mixing the components of any or all of the first through fourth liquids. Mixing is illustratively by convection, diffusion, or holographic optical tweezers wherein microspheres are spun in solution by holographically sculpted light fields. Illustratively as the result of convective and diffusive forces, a complementary element has an opportunity to be incorporated into the growing structure at the next hybridization or identification site. Similarly, the detecting template is mixed with solution containing or not containing the complementary recognition element species. Mixing illustratively occurs in a single recognition chamber or in separate areas of a recognition chamber or in a detection area. Alternatively, mixing occurs in an area intermediate between any chamber or portions of a chamber. In a preferred embodiment, a voltage potential is applied to the recognition chamber to move any unincorporated recognition elements through a surface or selective material to an area wherein detecting template is immobilized. A new round of recognition element is optionally added to the reactor area simultaneous to the prior recognition element polymerizing on a detecting template increasing the throughput of the sequencing identification process. Preferably, a buffer wash is performed in the reactor area during polymerization in the detection area. Also, during the polymerization reaction in the reactor area the detection area is optionally washed. This alternating cycle reduces the time required for sequence determination.

It is appreciated that any microdispenser is capable of dispensing any reagent or solution within the inventive apparatus and the order of addition to the recognition or other chamber is variable. Preferably, sequencing or detecting templates are deposited in the chambers prior to adding recognition elements or a single species of recognition element. Alternatively, microspheres with DNA polymerase are added to the recognition chamber followed by template to assemble the polymerization machinery and immobilize the template in position prior to addition of recognition element. These above examples are for illustrative purposes only, and it is appreciated that numerous other orders of addition are similarly operable in the inventive process.

An inventive apparatus also optionally comprises a collection area wherein synthesized strand is transferred from any chamber or other area of the apparatus for additional use of the structure molecules.

Most preferably, the reaction area contains no moving parts. Fluidic connection between each of the chambers is optionally powered by differential electric potential so as to move free recognition or building elements between the chambers. Further, DNA template and growing structure may similarly be transferred between chambers.

### Example 1

A standard reaction chamber protocol is outlined in FIG. 1C. DNA template with a known termination sequence of 3′-CAT TTT GCT GCC OGT CA- . . . -5′ (SEQ ID No. 1) is amplified by standard PCR techniques and purified on an anion exchange resin supplied by Quiagen, Inc., Valencia, Calif. 400 ng of template is added to each of four reaction chambers, a repeat detection chamber, and a sequence building chamber each containing a reaction solution of 60 nM Tris-SO₄ (pH 8.9), 180 mM Ammonium Sulfate. A primer (8 μg) of complementary sequence 5′-GTA AAA CGA CGG CCA GT-3′ (SEQ ID No. 2) is added to each chamber and allowed to hybridize with the DNA template under suitable conditions. A single species of Fluorecein-12 labeled A, T, G, and C nucleotides obtained from Perkin Elmer, Waltham, Mass. are added to each of the four reaction chambers 1/10 mol/mol concentration relative to DNA template. A polymerization reaction is initiated by the addition of 1 unit (final) of Platinum® Taq DNA Polymerase (Invitrogen, Inc., Carlsbad, Calif.) along with 2 mM MgSO₄ (final) in reaction solution. The reaction is allowed to proceed for 5 sec. An electric potential is applied to the solution of each reaction chamber in sequence whereby free nucleotide is selectively moved from the reaction chamber toward a detection area in which a fluorescent detector determines whether a fluorescent nucleotide is present in solution. Fluorescent parameters are 496 nm excitation, 517 nm emission with a 5 nm bandpass filter. Identification of which reaction chamber does not possess free labeled recognition element determines which element is next in sequence. The reaction chamber in which a labeled A was added demonstrates no free nucleotide.

Fluorescein-12 labeled A is added to the repeat detection chamber along with DNA polymerase, MgSO₄, and reaction solution by a microdispenser in 1/10 mol/mol amounts in sequential fashion and the reaction is allowed to proceed for 5 seq followed by application of an electric potential to determine if free nucleotide is present in solution. It is appreciated that other relative amounts of nucleotide and template are similarly suitable in all chambers. Application of an electric potential moves free nucleotide to a detector area where the presence of free nucleotide is determined as above. The process in the repeat detection chamber is repeated until free nucleotide recognized by the detector. Twenty additions are required for the instant exemplary template strand indicating that there is an AA repeat sequence.

2× mol/mol concentration of unlabeled A nucleotide (building element) is added to each of the reaction chambers and the sequence building chamber and the polymerization is allowed to proceed for 2 min followed by application of an electric potential to wash out any remaining free recognition or building element from all chambers.

The process is repeated for 350 cycles to fully assemble and identify the sequence of all nucleotide elements in the DNA sequence.

### Example 2

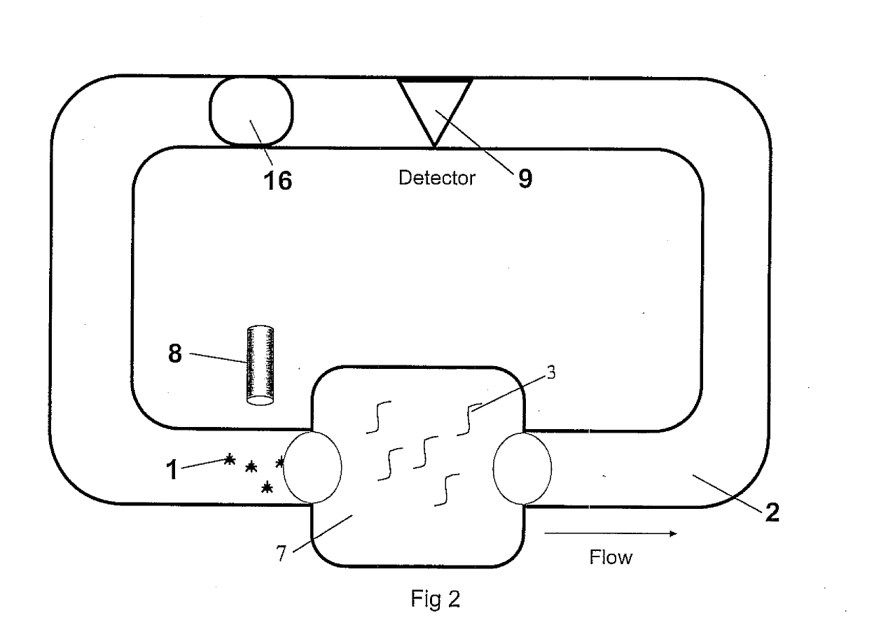

Referring to FIG. 2, a reaction chamber 2 is depicted as illustrated by a tubular loop structure wherein a support 7 is coated on a portion thereof which contains reaction solution. The support is coated with streptavidin by techniques known in the art. The primer of Example 1 is biotinylated by techniques known in the art illustratively by incorporation of biotin-aha-CTP (Invitrogen) in the primer sequence at the 5′ end. The primer is added to the reaction chamber and allowed to interact with the support. DNA template is then added at a concentration such that nearly all the DNA template will be hybridized with primer. Recognition elements 1, polymerase 5, and initiation ions such as in Example 1 are added by a microdispenser to the reaction chamber and a pump 16 circulates the fluid in the chamber such that the recognition element is flowed across the template bound support for 10 seconds with continuous monitoring by the fluorescent detector 9. Over the course of the reaction time the reaction chamber that contains the complementary recognition element demonstrates a reduction in fluorescence indicating that the element is incorporated onto the support bound primer. Analyses of each respective reaction chamber identify the next nucleotide in sequence. Two other similar reaction chambers, or standard container chambers are employed for the repeat detection chamber and the sequence building chamber and the structure building reaction and sequence identification is completed by subsequent iterative steps.

### Example 3

A DNA library is created by shear, immobilization, and amplification as described in Margulies, et al. 2005. Supports each containing a homogenous population of sequencing template copies are positioned in the wells of a sequencing micro-array such that one occupied well contains only one support and hence only one sequencing template sequence. The sequencing micro-array represents the recognition chamber and houses a reactor area and a detection area separated by fluidic connection or gel so that the supports will not be transferred from one area to another. The micro-array is placed between two cover slips raised off the outer surfaces of the array such that two flowcells are created with one on each side of the array. The detection area has in each well a plurality of supports each containing the entire family of detection template species. Thus, each detection area has all four, poly-A, poly-T, poly-G, and poly-C detection templates immobilized on the support.

The genomic library is amplified as briefly described in Margulies, et al., 2005 such that there are approximately 1×10⁷ copies of template DNA on each support. Thus, introduction of 1×10⁷ complementary recognition elements will occupy the next hybridization site on the growing structure on each support to fully saturate the next site. The detecting templates are each 50 elements in length and are present in a ratio of 1/50 the copy number of that in the reactor area.

Primers complementary to known sequence termination tags from the library along with Taq polymerase are moved though the flowcell on the reactor chamber side of the array. Similarly, four primers complementary to known sequences at a selected start of polymerization site just upstream of the oligomeric repeat region along with Taq polymerase as in Example 1 are applied into the flow chamber to load the wells of the detection chamber side of the array. Thus, the array is primed with the appropriate concentrations of polymerization enzyme, template, anions, and other necessary components for complementary replication of either sequencing template or detecting template.

A plurality of recognition elements in the same buffer solution as in Example 1, are placed in a reagent preparation area that is in fluidic connection with the flowcell on the reactor chamber side of the array. Each recognition element is labeled with a photocleavable 2-nitrobenzyl protecting group (Pillai, VNR, Synthesis, 1980; 2:1-26, the entire contents of which are incorporated herein by reference) and a fluorescent label that is distinguishable from the fluorescent labels of the other element species. Alternatively, the photocleavable fluorescent groups of Seo et al. are suitable fluorescent labels. Seo, T, et al., PNAS USA, 2005; 102:5926-31, the entire contents of which are incorporated herein by reference. However, noncleavable labels such as Cy5, Cy5, FITC, and Texas Red are employed so that the label is not sensitive to removal by the photocleavage reaction to remove the protecting group. Thus, each recognition element serves as a reversible terminator with a fluorescent label that allows distinction between the various species of elements. Ju et al., 2006 demonstrates the feasibility of DNA replication with photocleavable protecting groups on each of four nucleotides. Ju, J., et al., PNAS USA, 2006; 103:19635-19640, the entire contents of which are incorporated herein by reference.

All four labeled nucleotides are flowed over the wells of the reactor area and are passed into contact with the sequencing template and replication machinery including the primer by both convective and diffusive forces. After an appropriate amount of replication time an electric potential is applied to the entire surface of the plate to move all unbound recognition elements from the reactor areas to a series of individually dedicated detection areas wherein supports carrying detecting templates are present. The array is subjected to a Nd-YAG laser for 10 seconds to remove the photocleavable protecting group. As all replication machinery including the polymerase and primers are presenting the individual detecting areas, upon deprotection rapid incorporation of all free nucleotides occurs forming dsDNA.

In each well of the array a unique sequence is present. Thus, each well demonstrates chain extension of recognition elements in the detection area that were not next in sequence in the sequencing template. Differential fluorescence detection allows identification of the next incorporated element in each sequencing template without contamination of other template strands.

Following identification of the next element in sequence, the detection area is subjected to heat at 90° C. for 5 min to melt the DNA and washed in the flow chamber with buffer to remove the immobilized DNA strand, hence, regenerating the detection area.

The entire sequence is repeated for identification of the next element in series on the sequencing template. It is appreciated that while identification of as little as 7-26 elements in sequence is required for in silico construction of the genomic sequence, the instant process is repeated for 350 cycles for long chain sequence determination. Thus, rapid and inexpensive solution of whole genome sequence is achieved.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A process of self-assembling a number of elements into a structure comprising:
providing N recognition chambers,
adding a plurality of sequencing templates into a solution present in each chamber of said N recognition chambers;
adding a plurality of detecting templates into said solution present in each chamber of said N recognition chambers;
introducing a unique plurality of a homogeneous species of recognition elements to each chamber of said N recognition chambers;
exposing said plurality of templates and said unique plurality of recognition elements in each of said N recognition chambers to a polymerization reaction with a plurality of polymerization enzymes in each of said N recognition chambers;
identifying a next in sequence recognition element on said plurality of templates in at least one of said N recognition chambers;
repeating the introducing through subjecting steps until said structure is complete;
placing building elements corresponding to said next in sequence recognition element in at least one of said N recognition chambers; and
subjecting said plurality of templates and said building elements to said polymerization reaction in at least one of said N recognition chambers.

2. The process of claim 1, further comprising determining said template sequence.

3. The process of claim 1 wherein said recognition chamber further comprises a reactor area and a detection area.

4. The process of claim 3 wherein said detection template is immobilized in one of said detection area and said reactor area.

5. The process of claim 3 wherein a capture agent is immobilized in said detection area.

6. The process of claim 1 wherein said identifying a next in sequence recognition element further comprises detecting synthesis of complementary structure to said detection template.

7. The process of claim 1, further comprising:
placing in a repeat detecting chamber at least said plurality of sequencing templates and said solution wherein detecting repeat elements occurs by stepwise addition of building elements or recognition elements corresponding to said next in sequence recognition element;
transferring said solution from all N recognition chambers to said repeat detecting chamber prior to adding said building elements or recognition elements; and
calculating the number of repeat elements in the sequence of said template.

8. The process of claim 7, further comprising:
transferring said solution from said N recognition chambers and said solution from said repeat detection chamber to a sequence construction chamber; and
adding building elements corresponding to said next in sequence recognition element to said at least one of said N recognition chambers.

9. The process of claim 1 wherein said solution further comprises a primer sequence covalently hybridized to one of said plurality of sequencing templates or detecting templates.

10. The process of claim 1, wherein said unique plurality of recognition elements further comprise a label.

11. An apparatus for self-assembly of a number of elements comprising:
a reaction area;
a preparation area in fluidic connection with said reaction area;
a detection area in fluidic, physical, or optical connection with said reaction area;
said reaction area having no moving parts;
said reaction area having N recognition chambers;
each chamber having a plurality of microdispensers, each of said microdispensers capable of dispensing a unique species of recognition element or building element.

12. The apparatus of claim 11, where N is 4.

13. The apparatus of claim 11, further comprising a repeat detecting chamber.

14. The apparatus of claim 13, wherein said repeat detecting chamber is in fluidic connection with said recognition chambers and further comprising a sequence construction chamber in fluidic connection with said recognition chambers and said repeat detecting chamber.
